(19) United States
(12) Patent Application Publication
(10) Pub. No.: US 2026/0072017 A1
(43) Pub. Date: Mar. 12, 2026

(54) METHODS OF DETECTING OFF-TARGET BINDING OF PROTEINS

(71) Applicant: AMGEN INC., Thousand Oaks, CA (US)

(72) Inventors: Melissa YANG, Camarillo, CA (US); Marissa MOCK, Newbury Park, CA (US); René Simon HUBERT, Ventura, CA (US); Wenchao SUN, Oak Park, CA (US)

(73) Assignee: AMGEN INC., Thousand Oaks, CA (US)

(21) Appl. No.: 19/324,289

(22) Filed: Sep. 10, 2025

Related U.S. Application Data
(60) Provisional application No. 63/693,435, filed on Sep. 11, 2024.

(51) Int. Cl. G01N 33/53

(52) U.S. Cl. CPC G01N 33/5308; G01N 2430/60

(57) ABSTRACT

The present invention relates to methods of detecting off-target binding of proteins such as antibodies. Said methods are useful in identifying proteins that are likely to have more rapid clearance in vivo compared to proteins that do not have (or they have less) off-target binding.

### FIELD OF THE INVENTION

The present disclosure relates to methods of detecting off-target binding of proteins such as antibodies. Said methods are useful in predicting which proteins will have increased clearance in vivo.

### BACKGROUND OF THE INVENTION

During developability engineering, as a therapeutic progress through research to development and into the clinic, multiple analytical techniques are required for full characterization of the therapeutic to determine higher-order structure, performance in manufacturing processes, and the presence of any molecule-related impurities. Analytical techniques used for such purposes include, but are not limited to, size-exclusion chromatography (SEC), hydrophobic interaction chromatography (HIC), ion-exchange chromatography (IEX), gel- and capillary-electrophoresis (CE), differential scanning calorimetry (DSC), differential scanning fluorimetry (DSF), Fourier transform infrared spectroscopy (FT-IR), Fourier transform Raman spectroscopy (FT-Raman), protein nuclear magnetic resonance (NMR) spectroscopy, circular dichroism (CD), dynamic light scattering (DLS), rheology, mass spectrometry (MS), and tandem mass spectrometry (MS/MS). These methods are time-consuming since they are often manual and low throughput, and rely on the production and purification of recombinant protein samples.

Dostalek et al. and Hotzel et al. disclose non-specific binding assays including a baculovirus particles (BVP)-based ELISA assay (Dostalek et al., mAbs, 9 (5), 756-766 (2017) and Hotzel et al., mAbs, 4 (6), 753-760 (2012)). Hotzel et al. also discloses ELISA detection of non-specific binding to baculovirus particles that can identify antibodies having increased risk for fast clearance. However, methods with increased sensitivity and/or greater correlation with clearance are needed.

### SUMMARY OF THE INVENTION

The present invention provides a method of detecting off-target binding of a protein, said method comprising detecting binding of the protein to polyethyleneimine (PEI) or membrane preparation (MEM). In an embodiment, the method is an ELISA. In an embodiment, the method is a sandwich ELISA. In an embodiment, the method comprises coating a vessel with PEI or membrane preparation, and detecting binding of the protein to the PEI or membrane preparation. In an embodiment, binding is detected by reading absorbance at 450 nm. In an embodiment, off-target binding is detected if binding of the protein to PEI or membrane preparation is more than binding of the protein to an uncoated vessel. In an embodiment, the method comprises detecting binding of the protein to PEI. In an embodiment, the method comprises detecting binding of the protein to membrane preparation. In an embodiment, the protein binds to PEI or membrane preparation at least 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold more than the protein binds an uncoated vessel. In an embodiment, the protein binds to PEI or membrane preparation at least 3-fold more than the protein binds an uncoated vessel. In an embodiment, the protein binds to PEI or membrane preparation at least 4-fold more than the protein binds an uncoated vessel. In an embodiment, the protein binds to PEI or membrane preparation at least 14-fold more than the protein binds an uncoated vessel. In an embodiment, the protein is an antibody. In an embodiment, the method comprises detecting binding of the protein to PEI. In an embodiment, the method comprises detecting binding of the protein to MEM.

In an embodiment, a method of the present invention is used to predict clearance of a protein in vivo. In an embodiment, the binding of the protein to PEI or membrane preparation is detected, binding of the protein to an uncoated vessel is detected, and an increase in binding of the protein to PEI or membrane preparation compared to the protein binding to an uncoated vessel indicates that the protein would have increased clearance in vivo compared to a protein that has similar binding to PEI or membrane preparation and an uncoated vessel. In an embodiment, the method is used to predict clearance of a protein in mouse, cynomolgus monkey, or human. In an embodiment, the method is used to predict clearance of a protein in a mouse. In an embodiment, the method is used to predict clearance of a protein in cynomolgus monkey. In an embodiment, the method is used to predict clearance of a protein in a human. In an embodiment, the protein binds to PEI or membrane preparation at least 3-fold more than the protein binds an uncoated vessel. In an embodiment, the protein binds to PEI or membrane preparation at least 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold more than the protein binds an uncoated vessel. In an embodiment, the protein binds to PEI or membrane preparation at least 3-fold more than the protein binds an uncoated vessel. In an embodiment, the protein binds to PEI or membrane preparation at least 4-fold more than the protein binds an uncoated vessel. In an embodiment, the protein binds to PEI or membrane preparation at least 14-fold more than the protein binds an uncoated vessel. In an embodiment, clearance of the protein is determined. In an embodiment, there is increased binding of the protein to PEI or membrane preparation compared to binding of the protein to an uncoated vessel, and wherein the clearance is determined to be increased compared to a protein that has similar binding to PEI or membrane preparation and an uncoated vessel. In an embodiment, binding of the protein to PEI is detected. In an embodiment, binding of the protein to membrane preparation is detected. In an embodiment, the protein is an antibody. In an embodiment, the method comprises detecting binding of the protein to PEI. In an embodiment, the method comprises detecting binding of the protein to MEM.

In an embodiment, a method of the present invention is used to predict half-life of a protein in vivo. In an embodiment, the binding of the protein to PEI or membrane preparation is detected, binding of the protein to an uncoated vessel is detected, and an increase in binding of the protein to PEI or membrane preparation compared to the protein binding to an uncoated vessel indicates that the protein would have increased half-life in vivo compared to a protein that has similar binding to PEI or membrane preparation and an uncoated vessel. In an embodiment, the method is used to predict half-life of a protein in mouse, cynomolgus monkey, or human. In an embodiment, the method is used to predict half-life of a protein in a mouse. In an embodiment, the method is used to predict half-life of a protein in cynomolgus monkey. In an embodiment, the method is used to predict half-life of a protein in a human. In an embodiment, the protein binds to PEI or membrane preparation at least 3-fold more than the protein binds an uncoated vessel. In an embodiment, the protein binds to PEI or membrane preparation at least 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold more than the protein binds an uncoated vessel. In an embodiment, the protein binds to PEI or membrane preparation at least 3-fold more than the protein binds an uncoated vessel. In an embodiment, the protein binds to PEI or membrane preparation at least 4-fold more than the protein binds an uncoated vessel. In an embodiment, the protein binds to PEI or membrane preparation at least 14-fold more than the protein binds an uncoated vessel. In an embodiment, half-life of the protein is determined. In an embodiment, there is increased binding of the protein to PEI or membrane preparation compared to binding of the protein to an uncoated vessel, and wherein the half-life is determined to be increased compared to a protein that has similar binding to PEI or membrane preparation and an uncoated vessel. In an embodiment, binding of the protein to PEI is detected. In an embodiment, binding of the protein to membrane preparation is detected. In an embodiment, the protein is an antibody. In an embodiment, the method comprises detecting binding of the protein to PEI. In an embodiment, the method comprises detecting binding of the protein to MEM.

In an embodiment, 100 μl of PEI or membrane preparation are added to each well of a 96-well plate. In an embodiment, 0.1 μg/well of PEI or membrane preparation is added per well. In an embodiment, membrane preparation is CHEM-1 cell membrane preparation. In an embodiment, the PEI is PEI Max. In an embodiment, the method comprises adding horseradish peroxidase as a secondary antibody. In an embodiment, binding of the protein to PEI is detected. In an embodiment, binding of the protein to membrane preparation is detected. In an embodiment, the protein is an antibody.

### BRIEF DESCRIPTION OF THE DRAWINGS

FIG. 1 provides an overview of the format of ELISA assay to detect off-target binding and data from of a range of molecules.

### DETAILED DESCRIPTION

There is motivation to develop technologies that enable faster and more efficient therapeutic protein engineering. One approach is to build miniaturized and high (er) throughput versions of analytical techniques and use the measurements of molecule attributes such as colloidal, physical, chemical, and thermal stability to predict the developability of the protein.

Methods described herein are useful for predicting in vivo clearance (pharmacokinetics) and are useful to determine which proteins should or should not be pursued further (e.g. in cynomolgus monkey studies). Such methods include the use of substrates in off-target binding assays to screen for off-target binding of proteins. Coating substrates PEI (Polyethyleneimine Max-PEImax) and MEM (membrane prep from mammalian cell sources) are able to detect off-target binding, and are low cost and easy to be stored. The assays can be used as a method for high throughput analysis of off-target binding of proteins. ELISA assays using PEI or mouse cell membrane prep as coating substrates are used to detect off-target binding of proteins, and can be used to predict and screen for favorable pharmacokinetics of the protein. Interestingly, it was determined herein that protein binding to PEI or MEM showed a higher correlation with pharmacokinetics (PK) compared to protein binding to BVP.

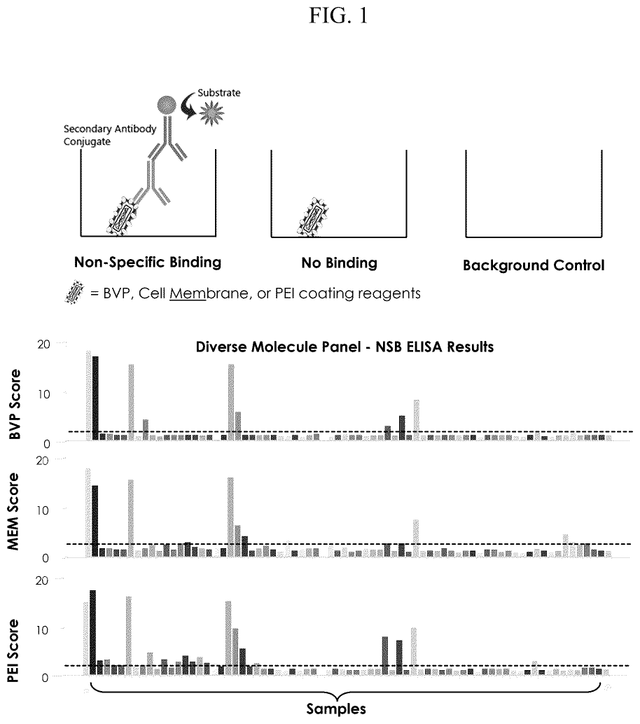

An enzyme-linked immunosorbent assay (ELISA) is a well-known vessel (plate)-based assay technique designed for detecting and quantifying soluble substances such as peptides, proteins, antibodies, and hormones. An ELISA can be performed according to methods known in the art and/or described herein including in the Examples. FIG. 1 is a schematic showing how off-target binding can be determined.

An ELISA relies on antibodies to detect a target antigen using highly specific antibody-antigen interactions. For example, a vessel can be coated with substrate (e.g. PEI or membrane prep) and incubated, protein is added, followed by addition of secondary antibody. The signal is then detected. It is envisaged that other ELISA formats can also be used as part of a method of the present invention. A vessel generally refers to containers. As used herein, a vessel refers to plates or wells that are well known to be used for ELISAs. Preferably, the vessel will have a clear top in order to read the emission. Specific ELISA plates are also commercially available and can be used.

In an ELISA assay, the antigen is immobilized to a solid surface. This is done either directly or via the use of a capture antibody itself immobilized on the surface. The antigen is then complexed to a detection antibody conjugated with a protein amenable for detection such as an enzyme or a fluorophore. An ELISA is typically performed in a multi-well plate (96- or 384-wells), which provides a solid surface to immobilize the antigen. In an ELISA, the antigen (target macromolecule) is immobilized on a solid surface (microplate) and then complexed with an antibody that is linked to a reporter enzyme. Detection is accomplished by measuring the activity of the reporter enzyme via incubation with the appropriate substrate to produce a measurable product.

There are several formats that may be used for ELISAs. The key step is immobilization of the antigen of interest, accomplished by either direct adsorption to the assay plate or indirectly via a capture antibody that has been attached to the plate. The antigen is then detected either directly (labeled primary antibody) or indirectly (such as labeled secondary antibody). A sandwich ELISA assay indirectly immobilizes and indirectly detects the presence of the target antigen. This type of capture assay is called a “sandwich” assay because the analyte to be measured is bound between two primary antibodies, each detecting a different epitope of the antigen—the capture antibody and the detection antibody. The sandwich ELISA format is highly used because of its sensitivity and specificity.

The direct detection method uses a primary antibody labeled with a reporter enzyme or a tag that reacts directly with the antigen. Direct detection can be performed with an antigen that is directly immobilized on the assay plate or with the capture assay format. Direct detection, while not widely used in ELISA, is quite common for immunohistochemical staining of tissues and cells.

An indirect detection method uses a labeled secondary antibody or a biotin-streptavidin complex for amplification. The secondary antibody has specificity for the primary antibody. In a sandwich ELISA, it is ideal that the secondary antibody is specific for the detection of the primary antibody only (and not the capture antibody) or the assay will not be specific for the antigen. Generally, this is achieved by using capture and primary antibodies from different host species (e.g., mouse IgG and rabbit IgG, respectively). For sandwich assays, it is beneficial to use secondary antibodies that have been cross-adsorbed to remove any secondary antibodies that might have affinity for the capture antibody.

Secondary antibodies that can be used in an ELISA are known in the art. If horseradish peroxidase (HRP) is used as a secondary antibody, the absorbance is read at 450 nm. However, a different absorbance can be used depending on the secondary antibody. For example, alkaline phosphatase can be read of 405 nm.

Protein binding to PEI or MEM can also be determined by binding assays commonly known in the art, including bead based alpha screens. For bead based alpha screens, beads can be coated with PEI or MEM and a proximity excitor will light up beads to which the protein binds. FACS-based techniques of binding analysis, yeast display, or phage display can also be used to determine binding of protein to PEI or MEM.

To “coat” a vessel (such as a plate or well), essentially refers to dispensing (e.g. pipetting) a reagent (e.g. substrate such as PEI or membrane prep) to the vessel. The substrate and vessel can then be incubated such that the substrate binds the vessel.

Engineered proteins such as antibodies are designed to bind at least one antigen (also referred to as a “target” or “intended target”). Multispecific proteins are designed to bind more than one target. An antibody contains complementarity-determining regions (CDR) in each of the heavy chains and light chains. The CDRs are highly responsible for binding of an antibody to its target. In order to increase the affinity of an antibody to its intended target, the CDRs may be altered (e.g. mutating certain amino acids), for example. However, despite engineering proteins to bind an intended target with high affinity, off-target binding can still occur for some proteins. Off-target binding refers to a protein (such as an antibody) that binds a molecule for which the protein was not engineered to bind. As used herein, this molecule is referred to as an “unintended target.” In an embodiment, a protein demonstrates off-target binding if it substantially binds (4-fold over control) to an unintended target(s).

Off-target binding can be determined by measuring the amount of binding of a protein to a coated vessel divided by the amount of binding of the protein to an uncoated vessel. The resulting is a normalized value represented as a fold increase. A cut-off can be determined, above which proteins are said to demonstrate off-target binding, and below which proteins are said to not demonstrate off-target binding. The cut-off can be set, in part, based on positive and negative controls. Briakinumab is an antibody that is known to have non-specific binding while Ustekinumab is an antibody that is known not to have non-specific binding. In addition, use of an automated liquid handler may result in less background signal, and a lower cut-off can be used (compared to a plate washer, for example, which could have higher background signal).

“Clearance” refers to a protein losing its presence in the body and is observed by the amount of protein in the body decreasing over time. One way a protein may be cleared is if it expelled or removed from the body. This can happen whether the protein has bound to its intended target or not. Another way a protein may be cleared is if the protein attaches to an unintended target and becomes sequestered with the unintended target, such that it can no longer bind to its intended target. Screening for off-target binding can help predict which proteins will have increased clearance via sequestration compared to proteins that do not demonstrate off-target binding. Half-life is the time it takes for half of the protein to be removed from the system. In an embodiment, a method of the present invention is used to correlate off-target binding and half-life of the protein.

Clearance can be determined by methods known in the art. For example, clearance can be quantified by measuring the amount of protein in blood, and calculating the area under the curve (AUC), the slope of the curve, and the half-life calculated from the curve.

If, for example, a panel of proteins are assessed for off-target binding by a method of the present invention, those proteins that demonstrate higher binding to PEI or MEM are expected to correlate with faster clearance (and increased half-life and poor PK) compared to proteins that demonstrate lesser binding (or no binding at all) to PEI or MEM. In this regard, and as used herein, determining binding of a protein to PEI or MEM is said to predict clearance or half-life of a protein. In an embodiment, binding of a protein to PEI or MEM is the only method used to predict clearance or half-life of a protein. In an embodiment, a Spearman Rank R>=0.4 is used to predict clearance. In an embodiment, a Spearman Rank R>=0.4 indicates a correlation between protein binding to PEI and clearance.

Correlation of off-target binding and clearance (or PK or half-life) can be determined by methods known in the art, including determining the Spearman Rank as described in Example 3 herein.

Polyethyleneimine (“PEI”) is a synthetic polymer with a linear or highly branched network and a high cationic charge density. It is commonly generated at molecular weights of 10, 25, and 40 kDa. PEI MAX (Polysciences) has a high density of protonatable amino groups, with amino nitrogen as every third atom.

Membrane preparation refers to lysing cells and centrifuging the lysed cells which then results in the cell membranes being separated in separate phases, thus enabling the cell membranes to be isolated from the rest of the cell contents. ChemiSCREEN Chem-1 Membrane Preparation is commercially available membrane preparation from Sigma-Aldrich. Chem-1 cells are an adherent cell line endogenously overexpressing Galpha15 that is used as a host for Chemicon's ChemiScreen GPCR Membrane Preparations.

A protein of a method of the present disclosure can be any protein that can bind a target. Non-limiting examples of proteins include antibodies, bispecific antigen-binding proteins, multispecific antigen-binding proteins, IgG-based proteins, IgG-scFv, IgG-Fab, IgG-scFab, BiTER molecules, and T-cell engagers. The protein should have an Fc if needed for secondary antibody binding. However, if the protein is a Fab, for example, a secondary antibody may be used that binds the protein in a region that is not the Fc.

As used herein, an “antibody” is an immunoglobulin molecule comprising 2 heavy chains (HCs) and 2 light chains (LCs) interconnected by disulfide bonds. The amino terminal portion of each LC and HC includes a variable region of about 100-120 amino acids primarily responsible for antigen recognition via the CDRs contained therein. The CDRs are separated with regions that are more conserved, termed framework regions (“FR”). Each LCVR and HCVR is composed of 3 CDRs and 4 FRs, arranged from amino-terminus to carboxy-terminus in the following order: FR1, CDR1, FR2, CDR2, FR3, CDR3, FR4. The 3 CDRs of the LC are referred to as “LCDR1, LCDR2, and LCDR3,” and the 3 CDRs of the HC are referred to as “HCDR1, HCDR2, and HCDR3.” The CDRs contain most of the residues which form specific interactions with the antigen. The functional ability of an antibody to bind a particular antigen is, thus, largely influenced by the amino acid residues within the six CDRs. Assignment of amino acids to CDR domains within the LCVR and HCVR regions of the antibodies of the present invention is based on the well-known Kabat numbering convention (Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)). It is understand that other numbering conventions may also be used, such as, for example, Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), and/or North (North et al., “A New Clustering of Antibody CDR Loop Conformations”, Journal of Molecular Biology, 406, 228-256 (2011)).

### EXAMPLES

### Example 1: Off-Target Binding

To determine off-target binding of antibody, ELISA plates (Costar 3590) were coated with 100 μl/well of 1) baculovirus particles (“BVP”, LakePharma 25,690) at 0.125 μg/well, 2) CHEM-1 cell membrane preparation (Millipore HTS000MC1) at 0.1 μg/well, 3) poly-D-Lysine (Millipore A-003-E) at 0.1 μg/well, or 4) PEI Max (Polysciences 24,765-2) at 0.1 μg/well. Plates were incubated at 4° C. overnight. Then the plates were blocked with SuperBlock™ T20 (PBS) Blocking Buffer (Thermo Scientific, Cat #37516) for 1 h at room temperature. The plates were washed three times with PBS. Proteins were diluted to 33 nM with SuperBlock™ T20 (PBS) Blocking Buffer. Triplicate aliquots of each test article were added to the plate wells. The plates were incubated for 1 hour at room temperature then were washed 6 times with PBS. 40 ng/ml of goat anti-human IgG conjugated to horseradish peroxidase (Jackson ImmunoResearch, Cat #109-035-008) was added to each well. The plates were incubated for 30 min at room temperature and then washed 6 times with PBS. TMB (3,3′,5,5′ tetramethylbenzidine) substrate (Thermo Scientific, Cat #34021) was added to each well. After incubation for 15 min, reactions were stopped by adding 2 M sulfuric acid. Absorbance was read at 450 nm using an AquaMax 4000 Spectrophotometer (Molecular Devices). The assay scores were generated by dividing the OD450 value of each well by the OD450 of a non-coated well.

According to these methods, 85 monoclonal antibodies (mAbs) were tested for off-target binding to BVP, PEI, and membrane preparation. Positive and negative controls were included. A normalized binding level (binding substrate/uncoated) of 14-fold was used. Of the 85 mAbs tested, one was found to demonstrate off-target binding to BVP. However, ten antibodies demonstrated off-target binding to PEI (Table 1; Sample number 5 also demonstrated off-target binding to BVP), and six antibodies demonstrated off-target binding to membrane preparation (Table 2). Five of the six antibodies that bound membrane preparation were the same as five of the ten antibodies that bound PEI.

TABLE 1
Binding values of mAbs (out of 85 tested) that demonstrate
above-threshold off-target binding to PEI.

| Sample No | Normalized Binding PEI |
|---|---|
| 5 | 18.5 |
| 74 | 16.3 |
| 88 | 15.9 |
| 94 | 16.9 |
| 102 | 27.6 |
| 109 | 16.0 |
| 112 | 18.8 |
| 118 | 33.2 |
| 119 | 26.8 |
| Positive control | 16 |

TABLE 2
Binding values of mAbs (out of 85 tested) that demonstrate
above-threshold off-target binding to membrane preparation.

| Sample No | Normalized Binding Membrane |
|---|---|
| 60 | 15.8 |
| 88 | 15.6 |
| 94 | 20.7 |
| 102 | 25.8 |
| 118 | 28.3 |
| 119 | 18.3 |
| Positive control | 16.3 |

Similar experiments were performed using an automated liquid plate handler and with a normalized binding cut-off of 4-fold. These data are shown in FIG. 1.

### Example 2: Coating Substrates

To determine binding of proteins to various substrates, vessels were coated with 10 ng/μl of the indicated substrates shown in Table 3. An ELISA was performed essentially as described in Example 1. Results are shown in Table 3 and are shown as a relative color comparison to BVP, which was set as ++.

Favorable coating reagents demonstrate signal with the positive control (an antibody known to have non-specific binding) and the absence of signal with the negative control (an antibody that is known not to have non-specific binding). Among the tested coating reagents, Membrane prep, Poly-D Lysine, hemoglobin, ssDNA and BVP showed detection of off-target binding.

TABLE 3
Binding of a monoclonal antibody to various coating substrates.

| | Binding to Positive | Binding to Negative |
|---|---|---|
| Coating Substrates | Control | Control |
| Heparin | − | − |
| Membrane Prep | ++ | − |
| Hemocyanin | − | − |
| PD-1 | − | − |
| Fibrinogen | + | + |
| Collagen | − | − |
| Poly-D Lysine | ++ | − |
| hemoglobin | ++ | − |
| No coating | − | − |
| Ovalbumin | − | − |
| Milk Powder | − | − |
| Cardiolipin Sodium Salt | − | − |
| ssDNA | + | − |
| dsDNA | − | − |
| Insulin | − | − |
| Transferrin | − | − |
| BVP | ++ | − |
| No coating | − | − |

### Example 3: Prediction of In Vivo Clearance

A total of 287 Fc containing molecules of various formats were selected to evaluate the correlation between attributes generated from in vitro assays and in vivo mouse PK clearance. The mouse PK clearance data have been generated historically. To generate the in vitro assay data, these molecules were recombinantly produced in CHO-X cells and purified by 1-step (for symmetric molecules) or 2-step (for asymmetric molecules) purification. 251 molecules passed quality control (QC) criteria that check the molecules' identity and purity. The purified material was formulated in 10 mM sodium acetate with 9% sucrose and the concentration was normalized to 5 mg/mL. A total of 21 analytical assays were performed using all or a subset of the 251 molecules. 31 attributes based on the output of one or more assays were selected to study the correlation with mouse total clearance. Spearman Rank correlation parameters were calculated using Spotfire Data Relationship tool with total clearance as Y and assay attributes as X. The scores for NSB PEI, NSB MEM and NSB BVP were included in the analysis.

Spearman rank correlation is obtained by first ranking all the values within each variables (NSB score and mouse total clearance (mL/hr/kg)) from lowest to highest, then calculating the Pearson correlation coefficient on those ranked values, essentially assessing the relationship between the ranks instead of the original data points. This allows for the analysis of non-linear monotonic relationships between variables.

To determine clearance, proteins were injected at the upper range of efficacious doses, and blood samples were taken periodically until the amount in serum got to below half of the starting dose and below the range of efficacy.

The mouse total clearance of all of the elimination processes in the body is calculated by dividing the dose administered by the total area under the plasma clearance time curve (AUC) from the time of dosing until drug concentrations can no longer be measured (CL=Dose/AUC).

The cut off >0.4 is selected based on the value commonly used in the literature to represent meaningful correlation. Eleven attributes had an absolute value of Spearman Rank R>=0.4, which means there is a correlation between attributes (e.g. binding PEI or MEM) and PK. Therefore, proteins that demonstrate off-target binding to PEI or MEM also have poor PK. Conversely, proteins that do not demonstrate off-target binding to PEI or MEM have better PK. The method described in Example 1 (and of the present invention) using PEI as a coating substrate showed the second highest predictivity for mouse PK as ranked by Spearman R correlations, and the method using membrane preparation (instead of PEI) was the tenth highest for predicting mouse PK. Binding to BVP did not have a Spearman Rank R>=0.4 and was therefore a worse predictor of PK compared to binding to PEI or MEM.

These data demonstrate that PEI and MEM are better predictors than BVP because it shows stronger correlation with a higher R value.

What is claimed:

1. A method of detecting off-target binding of a protein, the method comprising detecting binding of the protein to polyethyleneimine (PEI).

2. The method of claim 1, wherein the method is an ELISA.

3. The method of claim 1, the method comprising coating a vessel with PEI, and detecting binding of the protein to the PEI.

4. The method of claim 1, wherein binding is detected by reading absorbance at 450 nm.

5. The method of claim 1, wherein off-target binding is detected if binding of the protein to PEI is more than binding of the protein to an uncoated vessel.

6. The method of claim 1, the method comprising detecting binding of the protein to PEI.

7. The method of claim 1, wherein the method is a sandwich ELISA.

8. The method of claim 1 used to predict clearance of a protein in vivo, wherein binding of the protein to PEI is detected, binding of the protein to an uncoated vessel is detected, and an increase in binding of the protein to PEI compared to the protein binding to an uncoated vessel indicates that the protein would have increased clearance in vivo compared to a protein that has similar binding to PEI and an uncoated vessel.

9. The method of claim 8, in which the method is used to predict clearance of the protein in mouse, cynomolgus monkey, or human.

10. The method of claim 8, in which the method is used to predict clearance of the protein in a mouse.

11. The method of claim 8, in which the method is used to predict clearance of the protein in a cynomolgus monkey.

12. The method of claim 8, in which the method is used to predict clearance of the protein in a human.

13. The method of claim 8, wherein the protein binds to PEI at least 3-fold more than the protein binds an uncoated vessel.

14. The method of claim 8, wherein the protein binds to PEI at least 4-fold more than the protein binds an uncoated vessel.

15. The method of claim 8, wherein the protein binds to PEI at least 14-fold more than the protein binds an uncoated vessel.

16. The method of claim 8, wherein clearance of the protein is determined.

17. The method of claim 8, wherein there is increased binding of the protein to PEI compared to binding of the protein to an uncoated vessel, and wherein the clearance is determined to be increased compared to a protein that has similar binding to PEI and an uncoated vessel.

18. The method of claim 8, wherein the protein is an antibody.

19. The method of claim 8, wherein Spearman Rank is used to predict clearance.
